(19) United States
(12) Patent Application Publication
(10) Pub. No.: US 2017/0312517 A1
(43) Pub. Date: Nov. 2, 2017

(54) SYSTEM AND METHOD FOR TRAINING AND ASSESSMENT

(71) Applicant: HRL Laboratories, LLC, Malibu, CA (US)

(72) Inventors: Matthew E. Phillips, Calabasas, CA (US); Matthias Ziegler, Oakton, VA (US); Jaehoon Choe, Agoura Hills, CA (US)

(21) Appl. No.: 15/066,813

(22) Filed: Mar. 10, 2016

Related U.S. Application Data
(60) Provisional application No. 62/131,031, filed on Mar. 10, 2015.

(51) Int. Cl. A61N 1/36; G09B 9/24; A61N 1/04; A61B 5/00; A61B 5/0476; G09B 19/16

(52) U.S. Cl. CPC A61N 1/36025; G09B 19/165; G09B 9/24; A61B 5/0075; A61B 5/6803; A61B 5/0476; A61N 1/0456

(57) ABSTRACT

Described is a system for training and assessment. In operation, the system classifies a subject's baseline brain state and behavioral performance. Training goals are assessed to specify tasks the subject is to perform and a desired level of performance. The subject is subjected to neurological stimulation while the subject performs specified tasks. Behavioral data is assessed to determine if the subject has achieved the training goals. If the subject has achieved the training goals, the system stops. Alternatively, if the individual has not achieved the training goals, then neurological data is reviewed to identify activation states and values of the neurological stimulation that resulted in increased performance values from the baseline behavioral performance. The activation states and values of the neurological stimulation are adjusted to match those that resulted in increased performance values. The process is repeated until the subject has achieved the training goals.

### CROSS-REFERENCE TO RELATED APPLICATIONS

This is a non-provisional patent application of U.S. Provisional Application No. 62/131,031, filed on Mar. 10, 2015, the entirety of which is hereby incorporated by reference.

### BACKGROUND OF INVENTION

### (1) Field of Invention

The present invention is related to a training and assessment system and, more particularly, to a training system that assesses the effects of neurostimulation and training on real-world and abstracted task performance to improve subsequent training.

### (2) Description of Related Art

Pilot training and assessment is important in ensuring the quality and competence of airplane pilots. Current assessment metrics only exist for conventional pilot training methods; however, assessment systems do not currently exist for the evaluation of performance augmentation or identification of flight-subtask performance. Prior art in the pilot certification and assessment space exists in the form of written evaluations and other such conventional procedures, but these are often found to have poor correlation with real-world performance. For example, attempts to link test and questionnaire performance with practical flying have only yielded slight negative correlations of pilot judgement test (PJT) scores with number of incidents in aircraft on an annual basis (see the List of Incorporated References, Literature Reference No. 7). This type of correlation is neither exhaustive (does not cover general flying performance, only weighting flights that resulted in hazardous incidents) nor granular (all incidents are treated identically under this model).

Performance assessments for enhancements related to neurostimulation exist, but only in abstracted states unrelated to any discernible real-world task that correlate poorly to actual real-world performance (see, for example, Literature Reference Nos. 1 and 9). While some of these abstracted measures have been shown to predict performance by some investigators, others have not identified any such predictive power and this area can be considered an area of active research (see Literature Reference No. 9).

Further, existing performance enhancement/performance assessment modalities for neuro-augmentation consist of generalized intelligence tests, or testing of individual abilities, such as reaction times and working memory capacity. None of these metrics are directly related to practical flying tasks or real-world flight scenarios and have poor correlation with actual flying ability/skill (see Literature Reference No. 5). While abstracted task assessments such as that derived from intelligence tests or cognitive load “games,” can provide a rough estimate of task expertise and skill, correlation of abstracted intelligence measures and real-world scenarios do not provide a very dependable metric for practical execution of real-world tasks. This is true in a wide array of real-world scenarios, and is increasingly less reliable as the task becomes more complex (see Literature Reference Nos. 1, 3, 9, and 10).

Thus, a continuing need exists for a training and assessment system that (1) restructures the assessment procedure to a more performance-based and quantitative model; (2) identifies the specific, real-world improvements in functional performance that are directly related to the task at hand; and (3) provides a meaningful and practical feed-back and feed-forward source of performance data for use in brain-machine interface and performance-augmentation paradigms in the field of neurostimulation.

### SUMMARY OF INVENTION

This disclosure provides a training, system that assesses the effects of neurostimulation and training on real-world and abstracted task performance to improve subsequent training. The system includes one or more processors and a memory. The memory is a non-transitory computer-readable medium having executable instructions encoded thereon, such that upon execution of the instructions, the one or more processors perform several operations, including classifying a subject's baseline brain state and behavioral performance; assessing training goals to specify which tasks the subject is to perform and a desired level of performance; subjecting the subject to neurological stimulation while the subject performs specified tasks; and assessing behavioral data to determine if the subject has achieved the training goals.

In another aspect, in assessing behavioral data to determine if the subject has achieved the training goals, if the subject has achieved the selected goal, then stopping.

Further, in assessing behavioral data to determine if the subject has achieved the training goals, if the individual has not achieved the training goals, then performing operations of reviewing neurological data to identify activation states and values of the neurological stimulation that resulted in increased performance values from the baseline behavioral performance; adjusting the activation states and values of the neurological stimulation to match those that resulted in increased performance values; and repeating the operations of subjecting the subject to neurological stimulation, assessing behavioral data, and reviewing the neurological data to identify activation states and values until the subject has achieved the training goals.

Additionally, in reviewing neurological data to identify activation states and values of the neurological stimulation, the neurological data is at least one of functional near-infrared spectroscopy imagery and electroencephalogram data.

In another aspect, in assessing behavioral data, the behavioral data includes performance data.

In yet another aspect, the system includes an elastic headcap having a plurality of sensors and stimulators.

In yet another aspect, the system for training and assessment is a pilot training and assessment system, such that when the subject performs the specified tasks, the specified tasks are performed in a flight simulator, with the behavioral data being flight data as recorded by the flight simulator.

Finally and as noted above, the present invention also includes a computer program product and a computer implemented method. The computer program product includes computer-readable instructions stored on a non-transitory computer-readable medium that are executable by a computer having one or more processors, such that upon execution of the instructions, the one or more processors perform the operations listed herein. Alternatively, the computer implemented method includes an act of causing a computer to execute such instructions and perform the resulting operations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The objects, features and advantages of the present invention will, be apparent from the following detailed descriptions of the various aspects of the invention in conjunction with reference to the following drawings, where:

FIG. 1 is a block diagram depicting the components of a system according to various embodiments of the present invention;

FIG. 2 is an illustration of a computer program product embodying an aspect of the present invention;

FIG. 3A is a flowchart depicting a process for training and assessment according to various embodiments of the present invention;

FIG. 3B is an illustration of a headcap according to various embodiments of the present invention;

FIG. 3C is an illustration depicting examples of possible configurations for implementing the training system, including example sensor and stimulator locations and corresponding modeling given the example sensor and stimulator locations;

FIG. 4 is a flowchart depicting training and assessment processes;

FIG. 5 is an illustrating depicting an example of cognitive testing procedures;

FIG. 6 is a table providing example flight testing protocol tasks

FIG. 7A is an illustration depicting training behavioral data and fNIRS imaging for unstimulated subjects on day 1 of training;

FIG. 7B is an illustration depicting training behavioral data and fNIRS imaging for unstimulated subjects on day 4 of training;

FIG. 7C is an illustration depicting training behavioral data and fNIRS imaging for stimulated subjects on day 1 of training; and

FIG. 7D is an illustration depicting training behavioral data and fNIRS imaging for stimulated subjects on day 4 of training.

### DETAILED DESCRIPTION

The present invention is related to a training and assessment system and, more particularly, to a training system that assesses the effects of neurostimulation and training on real-world and abstracted task performance. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein.

In the following detailed description, numerous specific details are set forth in order to provide a more thorough understanding of the present invention. However, it will be apparent to one skilled in the an that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Before describing the invention in detail, first a list of cited references is provided. Next; a description of the various principal aspects of the present invention is provided. Subsequently, an introduction provides the reader with a general understanding of the present invention. Finally, specific details of various embodiment of the present invention are provided to give an understanding of the specific aspects.

### (1) LIST OF CITED LITERATURE REFERENCES

The following references are cited throughout this application. For clarity and convenience, the references are listed herein as a central resource for the reader. The following references are hereby incorporated by reference as though fully set forth herein. The references are cited in the application by referring to the corresponding literature reference number.

1. Alloway, T. P., and Alloway, R. G. “Investigating the predictive roles of working memory and IQ in academic attainment.” Journal of experimental child psychology 106.1 (2010): 20-29.

2. Ayaz, H., Bunce, S., Shewokis, P., Izzetoglu, K., Willems, B., and Onaral, B. “Using brain activity to predict task performance and operator efficiency.” Advances in Brain inspired Cognitive Systems. (2012): 147-155.

3. Ehrman, M. “A Study of the Modern Language Aptitude Test for Predicting Learning Success and Advising Students.” Language Aptitude Invitational Symposium Program Proceedings. Arlington, Va., Sep. 25-27, 1994.

4. Ford, J. Kevin, and Douglas Sego. “Linking training evaluation to training needs assessment: A conceptual model.” Michigan State Univ East Lansing Dept Of Psychology, 1990.

5. Gott, Sherrie P. “Rediscovering learning: acquiring expertise in real world problem solving tasks,” No. AL/HR-TP-1997-0009. Armstrong Lab Brooks AFB, TX Human Resources Directorate, 1998.

6. Harrison, J., Izzetoglu K., Ayaz, H., Willems. B., Hab, S., Ahlstrom, U., and Onaral, B. “Cognitive Workload and Learning Assessment During the Implementation of a Next-Generation Air Traffic Control Technology Using Functional Near-Infrared Spectroscopy.” (2014)

7. Hunter, D. R. “Measuring general aviation pilot judgment using a situational judgment technique.” The International Journal of Aviation Psychology 13, no. 4 (2003): 373-386.

8. Hunter. D. R. “Airman Research Questionnaire, Methodology and Overall Results.” No. DOT/FAA/AM-95/27. Federal Aviation Administration Washington Dc Office Of Aviation Medicine, 1995.

9. Redick, T. S. et al. “No evidence of intelligence improvement after working memory training: a randomized, placebo-controlled study.” Journal of Experimental Psychology: General 142.2 (2013): 359.

10. Sohn, Y. W., and Doane, S. M. 2004. “Memory Processes of Flight Situation Awareness: Interactive Roles of Working Memory Capacity, Long-Term Working Memory, and Expertise.” Human Factors: Journal of the Human Factors and Ergonomics Society 46 (3): 461-75.

(2) Principal Aspects

Various embodiments of the invention include three “principal” aspects. The first is a training (e.g., pilot training) and assessment system that assesses the effects of neurostimulation and training on real-world and abstracted task performance (e.g., during flight) and, based on such assessments, modifies the training program. The system is typically in the form of a computer system (having one or more processors) operating software or in the form of a “hard-coded” instruction set. This system may be incorporated into a wide variety of devices that provide different functionalities. The second principal aspect is a method, typically in the form of software, operated using a data processing system (computer). The third principal aspect is a computer program product. The computer program product generally represents computer-readable instructions stored on a non-transitory computer-readable medium such as an optical storage device, e.g., a compact disc (CD) or digital versatile disc (DVD), or a magnetic storage device such as a floppy disk or magnetic tape. Other, non-limiting examples of computer-readable media include hard disks, read-only memory (ROM), and flash-type memories. These aspects will be described in more detail below.

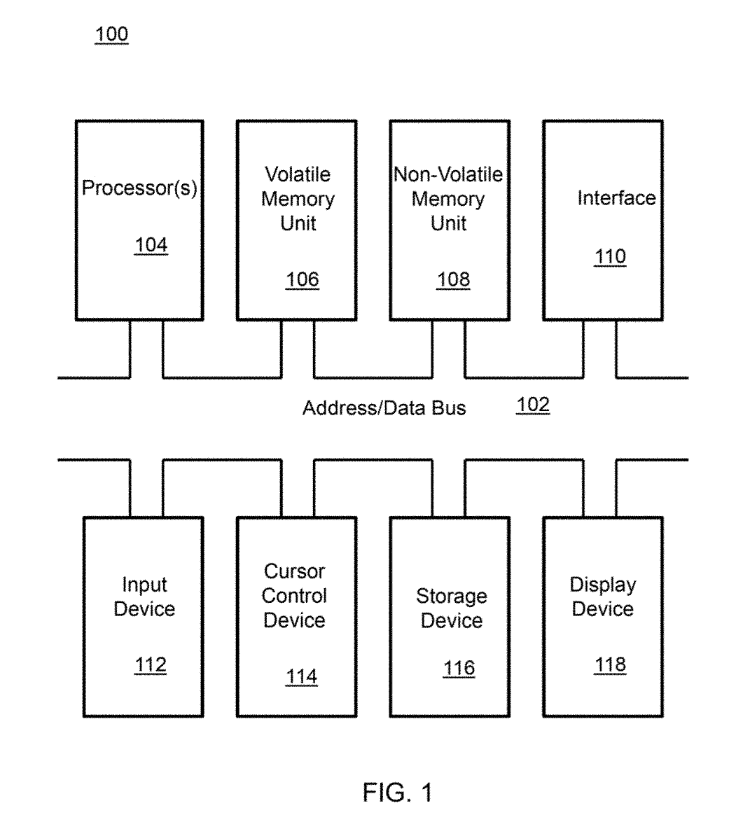

A block diagram depicting an example of a system (i.e., computer system 100) of the present invention is provided in FIG. 1. The computer system 100 is configured to perform calculations, processes, operations, and/or functions associated with a program or algorithm. In one aspect, certain processes and steps discussed herein are realized as a series of instructions (e.g., software program) that reside within computer readable memory units and are executed by one or more processors of the computer system 100. When executed, the instructions cause the computer system 100 to perform specific actions and exhibit specific behavior, such as described herein.

The computer system 100 may include an address/data bus 102 that is configured to communicate information. Additionally, one or more data processing units, such as a processor 104 (or processors), are coupled with the address/data bus 102. The processor 104 is configured to process information and instructions. In an aspect, the processor 104 is a microprocessor. Alternatively, the processor 104 may be a different type of processor such as a parallel processor, or a field programmable gate array.

The computer system 100 is configured to utilize one or more data storage units. The computer system 100 may include a volatile memory unit 106 (e.g., random access memory (“RAM”), static RAM, dynamic. RAM, etc.) coupled with the address/data bus 102, wherein a volatile memory unit 106 is configured to store information and instructions for the processor 104. The computer system 100 further may include a non-volatile memory unit 108 (e.g., read-only memory (“ROM”), programmable ROM (“PROM”), erasable programmable ROM (“EPROM”), electrically erasable programmable ROM “EEPROM”), flash memory, etc.) coupled with the address/data bus 102, wherein the non-volatile memory unit 108 is configured to store static information and instructions for the processor 104. Alternatively, the computer system 100 may execute instructions retrieved from an online data storage unit such as in “Cloud” computing, In an aspect, the computer system 100 also may include one or more interfaces, such as an interface 110, coupled with the address/data bus 102. The one or more interfaces are configured to enable the computer system 100 to interface with other electronic devices and computer systems. The communication interfaces implemented by the one or more interfaces may include wireline (e.g., serial cables, modems, network adaptors, etc.) and/or wireless (e.g., wireless modems, wireless network adaptors, etc.) communication technology.

In one aspect, the computer system 100 may include an input device 112 coupled with the address/data bus 102, wherein the input device 112 is configured to communicate information and command selections to the processor 100. In accordance with one aspect, the input device 112 is an alphanumeric input device, such as a keyboard, that may include, alphanumeric and/or function keys. Alternatively, the input device 112 may be an input device other than an alphanumeric input device. In an aspect, the computer system 100 may include a cursor control device 114 coupled with the address/data bus 102, wherein the cursor control device 114 is configured to communicate user input information and/or command selections to the processor 100. In an aspect, the cursor control device 114 is implemented using a device such as a mouse, a track-ball, a track-pad, an optical tracking device, or a touch screen. The foregoing notwithstanding, in an aspect, the cursor control device 114 is directed and/or activated via input from the input device 112, such as in response to the use of special keys and key sequence commands associated with the input device 112. In an alternative aspect, the cursor control device 114 is configured to be directed or guided by voice commands.

In an aspect, the computer system 100 further may include one or more optional computer usable data storage devices, such as a storage device 116, coupled with the address/data bus 102. The storage device 116 is configured to store information and/or computer executable instructions. In one aspect, the storage device 116 is a storage device such as a magnetic or optical disk drive (e.g., hard disk drive (“HDD”), floppy diskette, compact disk read only memory (“CD-ROM”), digital versatile disk (“DVD”)). Pursuant to one aspect, a display device 118 is coupled with the address/data bus 102, wherein the display device 118 is configured to display video and/or graphics. In an aspect, the display device 118 may include a cathode ray tube (“CRT”), liquid crystal display (“LCD”), field emission display (“FED”), plasma display, or any other display device suitable for displaying video and/or graphic images and alphanumeric characters recognizable to a user.

The computer system 100 presented herein is an example computing environment in accordance with an aspect. However, the non-limiting example of the computer system 100 is not strictly limited to being a computer system. For example, an aspect provides that the computer system 100 represents a type of data processing analysis that may be used in accordance with various aspects described herein. Moreover, other computing systems may also be implemented. Indeed, the spirit and scope of the present technology is not limited to any single data processing environment. Thus, in an aspect, one or more operations of various aspects of the present technology are controlled or implemented using computer-executable instructions, such as program modules, being executed by a computer. In one implementation, such program modules include routines, programs, objects, components and/or data structures that are configured to perform particular tasks or implement particular abstract data types. In addition, an aspect provides that one or more aspects of the present technology are implemented by utilizing one or more distributed computing environments, such as where tasks are performed by remote processing devices that are linked through a communications network, or such as where various program modules are located in both local and remote computer-storage media including memory-storage devices.

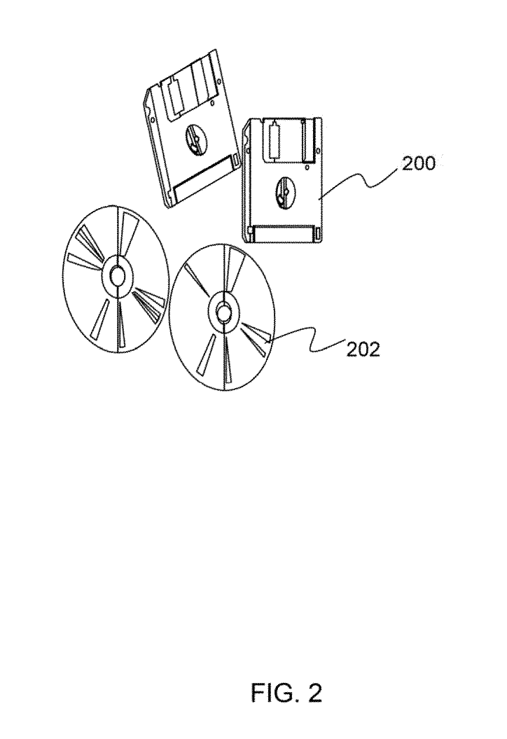

An illustrative diagram of a computer program product (i.e., storage device) embodying an aspect of the present invention is depicted in FIG. 2. The computer program product is depicted as floppy disk 200 or an optical disk 202 such as a CD or DTD. However, as mentioned previously, the computer program product generally represents computer-readable instructions stored on any compatible non-transitory computer-readable medium. The term “instructions” as used with respect to this invention generally indicates a set of operations to be performed on a computer, and may represent pieces of a whole program or individual, separable, software modules. Non-limiting examples of “instruction” include computer program code (source or object code) and “hard-coded” electronics (i.e. computer operations coded into a computer chip). The “instruction” is stored on any non-transitory computer-readable medium, such as in the memory of a computer or on a floppy disk, a CD-ROM, and a flash drive. In either event, the instructions are encoded on a non-transitory computer-readable medium.

(3) Introduction

This disclosure provides a system and method to assess the effects of neurostimulation and training on real-world and abstracted task performance (such as those performed during flight by a pilot). The system analyzes the complexities of real-world task performance directly rather than relying purely on abstracted data to infer skill and expertise. The system uses a multi-modal set of measurements including recordings that determine brain states and neurological function, standard workload assessment tests, and the quantification and analysis of task-relevant, real-world behavioral data. These different sets of data allow the system to gauge pilot skill and performance with precision and specificity beyond that of conventional assessment procedures.

By assessing key quantitative elements of the actual task of interest and relating these data back to defined brain states and stimulation patterns, the system is able to determine the effects of performance-augmenting neurostimulation and specialized training methods on skills of interest directly. The method described herein is better able to pair desirable task skill levels with quantifiable diagnostic data taken during training sessions and task execution and, thereby achieve greater overall predictive and prescriptive power over real-world performance of pilot trainees. In other words, based on the initial assessments, subsequent training can be implemented incorporating the neurostimulation or behavioral tasks that provided the best results.

As can be appreciated by those skilled in the art, the system described herein provides several applications and benefits. For example, the system can be used to assess the effects of human augmentation in relation to practical, real-world tasks and provides critical feed-back and feed-forward data for modulation and modification of stimulation protocols. This capability is essential for the construction of advanced neuroaugmentation technologies and the development of control-theory-based brain-machine interface systems and can “slot-in” easily to non-flight related paradigms and technologies.

For pilot training, the multi-modal, task-specific analytics system provides a more accurate picture of pilot training status and knowledge. The invention enables the accurate definition of pilot strengths and weaknesses and can provide guidance to determine the specific types of training from which a pilot could maximally benefit. This has broad applications for pilot candidacy determination, increased efficiency of existing training regimes, and could aid the guided development of new training procedures, all of which drastically reduce the resources and expenses that are typically associated with advanced pilot training. It should be noted that although pilot training is reference and used herein as an example, the system and method are not limited thereto as it can be used for any trainable skill that, during operation, results in the performance of tasks. Other non-limiting examples of such domains include second language acquisition, leadership training, and intelligence analysis, all of which include multi-modal skillsets as a part of task proficiency and can benefit from specific, accurate delineation of candidate strengths and weaknesses to better inform training strategies and systems.

(4) Specific Details of Various Embodiments

As noted above, this disclosure provides a system and method to assess the effects of neurostimulation and training on real-world and abstracted task performance. The obtained neurological data and behavioral data are then used as prescriptions to change the stimulation (e.g., electrode placement or values) and/or task to improve the training performance.

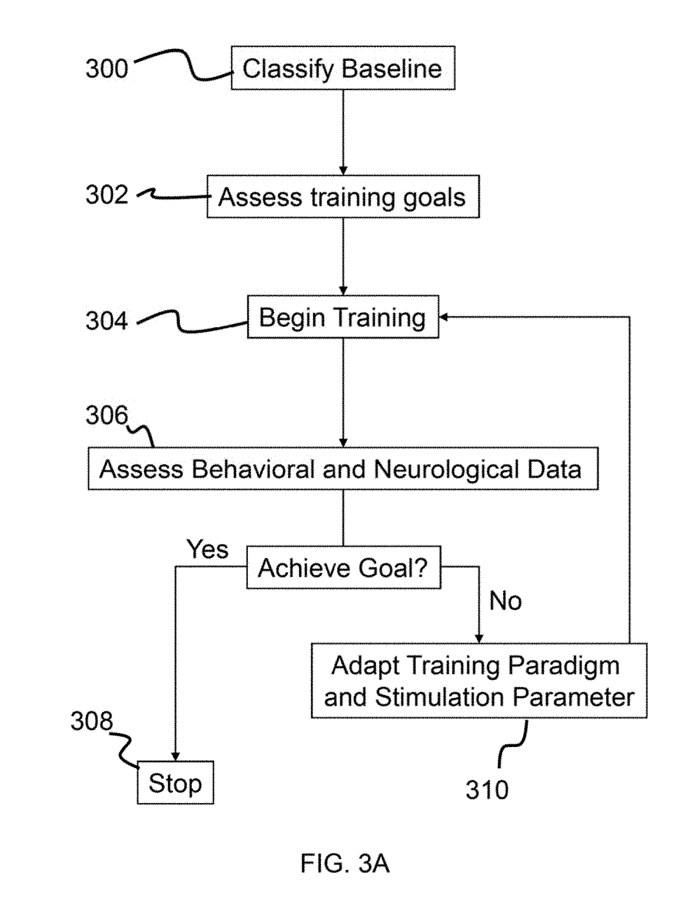

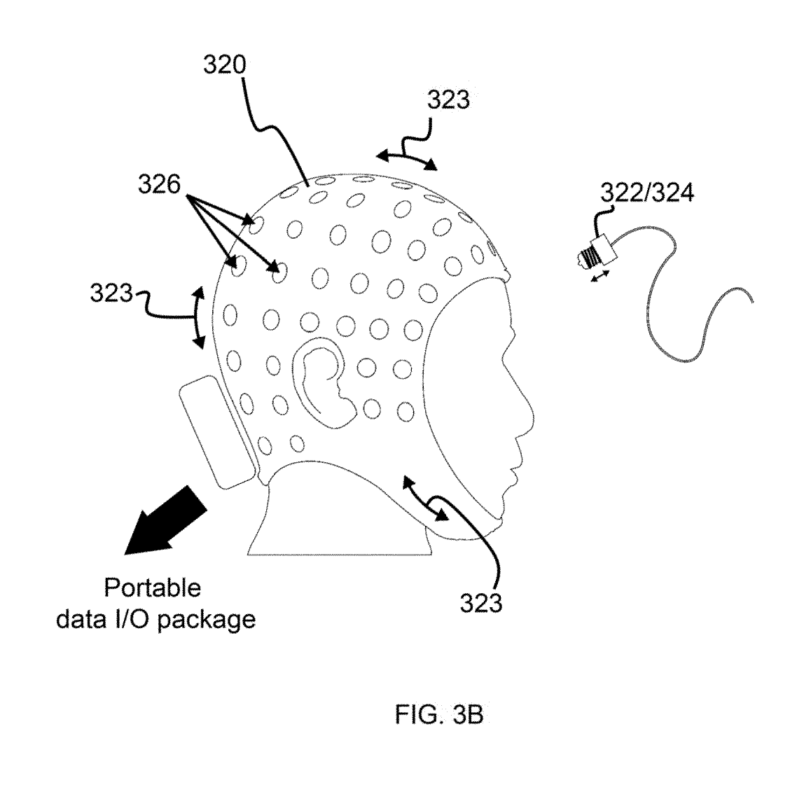

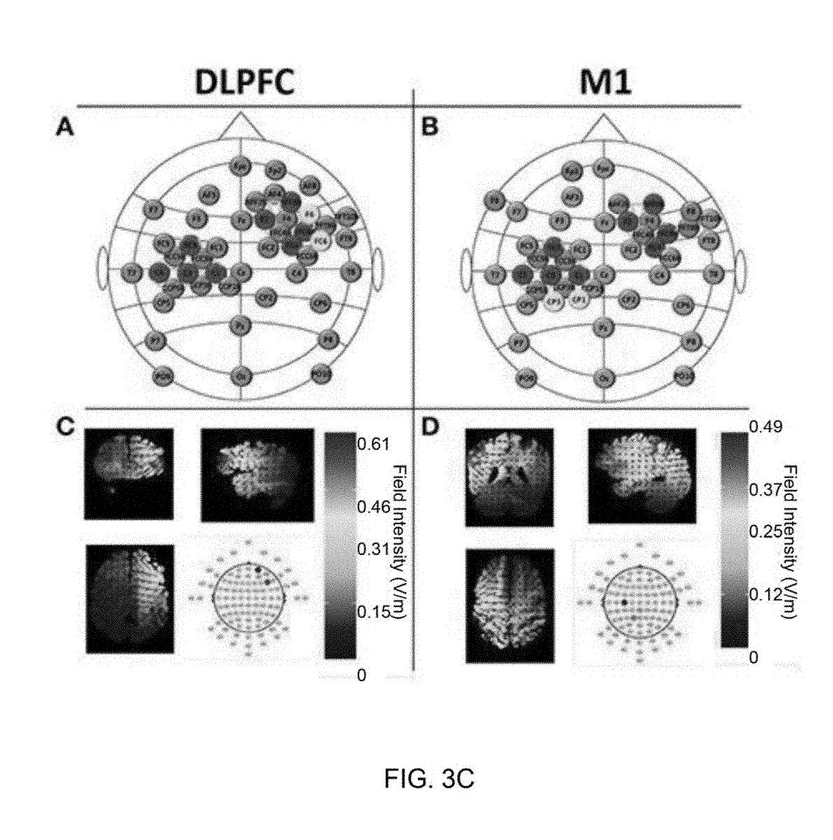

For example and as shown in FIG. 3A, the system can be implemented to adapt training paradigms and stimulation parameters. In order to do so, the system must be used to classify 300 the subject's (i.e., trainee or person) baseline brain state (e.g., neurological data) and behavioral performance (e.g., behavioral data). In other words, the brain activity sensors (as described below) are attached with the subject and the subject is asked to perform a certain task. The system monitors the subject's neurological data and behavioral data to calibrate the system with a pre-training level of performance on the task for that subject.

Training goals are assessed 302 to determine which tasks to perform and train and a desired level of improvement or performance. For example, if during calibration it is determined that the subject performs a particular task at a skill level 2, it may be desired that the subject perform that task at a skill level 6. Thus, in this non-limiting example, the training goal may be automatically set or input by an operator of increasing task performance from skill level 2 to skill level 6.

With training goals specified, the training 304 can begin. As depicted in the example shown in FIG. 3B, the subject may be taught and/or asked to perform the selected skill/task while being subjected to neurological stimulation via headgear, such, as a headcap 320 containing both: 1) sensors 322 to detect high-resolution spatiotemporal neurophysiological activity; and 2) a montage of stimulation elements that can be used to direct current flow to specific cortical subregions. should be understood that additional headgear configurations can also be implemetnted so long as they include the sensors and stimulation eleements, additional non-limiting examples include a non-elastic headcap, nets (such as hair or head nets), bands, visors, helmets, or other headgear, etc.

In some embodiments, the headcap 320 is formed of an elastic material containing sensing components that record neurophysiological activity via electrical potentials on the scalp (electroencephalogram (EEG)) and backscattered near infrared light detecting conical bloodflow (functional near-infrared spectroscopy, FNIRS). Both sensors may need to be present in the cap in order to delineate cortical activity at high spatial and temporal resolution, and the headcap is elastic (compression fitting 323) to fixate the sensitive recording elements to ensure the procurement of dean, artifact-free signals to feed the training system (and to provide for sensor and stimulator consistency). Stimulation elements 324 are also present in the same headcap 320 device, which includes multiple sets of surface electrodes which are precisely controlled to direct currents through the scalp and interstitial tissues to cortical regions of interest (high-definition transcranial current stimulation (HD-tCS)). In some embodiments, these stimulation elements 324 maintain consistent electrical environments—particularly impedance values—in order to provide appropriate stimulation throughout training. The control software of the electrodes also enables the modification of the injected electrical current, as varying stimulation protocols can be leveraged to achieve differential effects to neurological tissue. In the same vein, the headcap 320 itself in some embodiments is configurable—that is, the headcap 320 is constructed such that all sensing and recording components have modular configurability to allow recordings to be taken from diverse areas of the scalp, and stimulation to be applied to a wide array of brain structures. For example, the headcap 320 is depicted as having a plurality of configurable harness locations 326 for receiving a sensor 322 and/or stimulator 324. The sensors 322 and stimulators 324 can be formed and combined in a single harness for attaching at a harness location 326 or they can be separately attached. The sensors 322 and stimulators 324 may also be spring-loaded to maintain sufficient contact with the wearer's skin. For various embodiments, one, some, or all of these components are present in the headcap 320, and these characteristics of the device are helpful for the application of Neurostimulation-based training and performance assessment.

Neurological stimulators (electrodes) are attached to the brain (via, for example, the headcap) and turned on and/or activated at different values (e.g., different waveform values). For example, to improve behaviors involving reasoning and executive function (as directed by 306), the system targets the dorsolateral prefrontal cortex (DLPFC) as denoted by the purple and yellow markers (cathodes and anodes, respectively) in FIG. 3C. Specifically, FIG. 3C is an illustration depicting examples of possible configurations for implementing the training system, including example sensor and stimulator locations and corresponding modeling given the example sensor and stimulator locations. Boxes A and C refer to the DLPFC while Boxes B and D refer to the primary motor cortex (M1). Box A depicts example sensor and stimulator configurations for DLPFC stimulation, while Box B depicts example sensor and stimulator COD figurations for the M1 stimulation. The Blue markers are. EEG probes (recording of electrical activity) while the Red and Green markers are fNIRS optodes (bloodflow/blood oxygenation recording; i.e. real-time brain metabolism monitoring). The red and green are infrared light sources and sensors, respectively. Thus, Boxes A and Bin FIG. 3C provide non-limiting examples of two possible configurations for facilitating the training system.

Box C of FIG. 3C depicts finite elements modeling (FEM) of the electrical current flow for the configuration of electrodes given in Box A. This physics simulation is used in order to predict where the current from the stimulation electrodes will end up inside the brain. It is shown that the intended target is the DLPFC, which holds up in the simulation.

Box D of FIG. 3C depicts finite elements modeling (FEM) of the electrical current flow for the configuration of electrodes given in Box B. This physics simulation is used in order to predict where the current from the stimulation electrodes will end up inside the brain. Again, the prediction given by the simulation matches the target (i e., M1). Thus, Boxes C and D demonstrate that modular and configurable stimulation probes are essential for targeting different regions of the brain. As noted above, a non-limiting example of the modular nature of stimulation/recording components of the headcap is shown in FIG. 3B. For example, all locations have the capability to be arranged in specific configurations that monitor and target selected cortical subregions.

For continuous, individualized adaptation and quantification of training progress, the above described headcaps provide, neurophysiological state data, which provide quantitative metrics to identify the current state of training for subjects and identify trainee phenotypes that describe individualized performance capabilities and possible training outcomes. These models are used to generate and update individualized training regimes that predict optimal stimulation parameters and behavioral training to effect changes in the brain most advantageous for a given task. Pre-existing templates of domain experts and adaptation mechanisms generated from recorded neurological data are used to compute the most likely stimulation parameters and brain states are then “guided” towards a calculated optimum through personalized Neurostimulation and training. Hence, the system operates in a closed loop in which incoming neurophysiological data is used to update software models of user state, that then calculate the optimal intervention, which is conveyed to the stimulation components to provide neuromodulation at prescribed intervals, locations, and intensities.

Thus, the behavioral and neurological data is continually assessed 306 to determine the performance parameters of the subject and determine if the subject has achieved the selected training goal. If the subject has achieved the selected training goal, then the training process stops 308 for the selected task. Alternatively, if the subject has not achieved the selected training goal, then the system identifies the activation states (e.g., on/off) and values of the neurological stimulators that resulted in increased performance values from the baseline and subsequent assessments. Based on that, the system adapts 310 the training paradigm and stimulation parameter to adjust the activation states and values of the neurological stimulators (i.e., to match those that resulted in increased performance). Training 304 is then continued, with the process repeating until the subject has achieved the selected training goal. These aspects are described in further detail below.

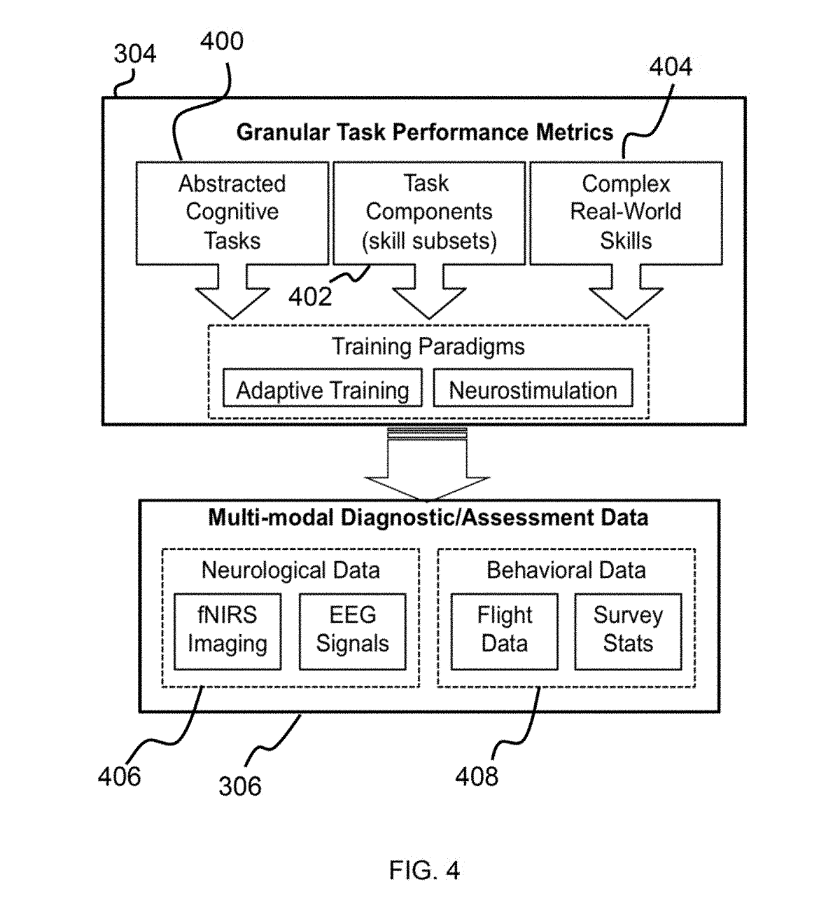

As shown in FIG. 4, training 304 includes three broad task assessment sources, abstracted cognitive tasks 400, task components 402, and complex real-world skills 404, each of which is addressed in turn below.

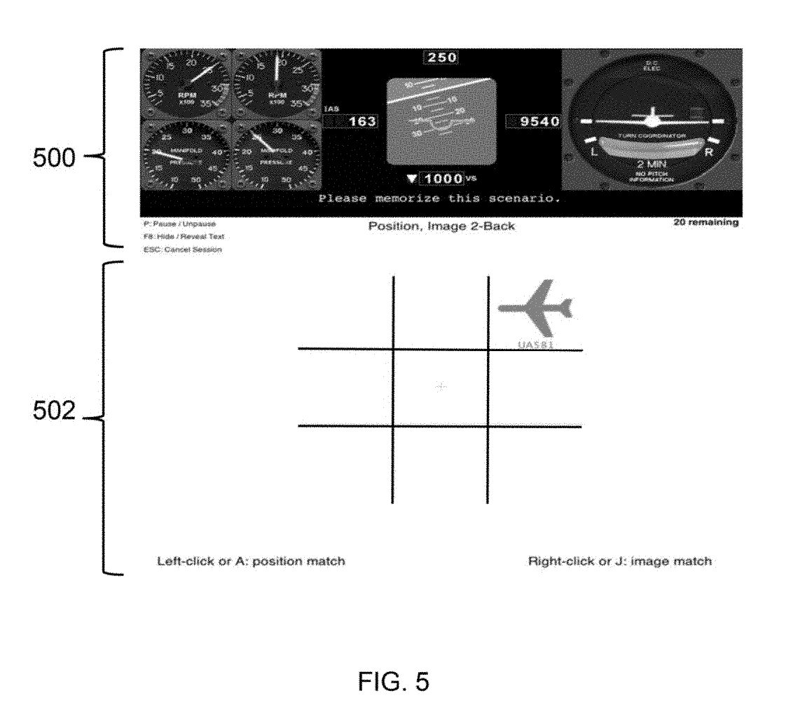

The abstracted cognitive task 400 phase of performance assessment resembles tasks typically used to determine the effects of neurostimulation on cognitive subcomponents, such as fluid intelligence, working memory, and memory retrieval. These tests are primarily used to obtain basic cognitive data analogous to that described in existing literature and documentation in order to find correlates, if any, of basic cognitive subcomponent performance with real-world skill acquisition and expertise. These tasks 400, for example, were modeled from a Working Memory Task (the “n-Back task,” as described in Literature Reference No. 2), and a Situational Awareness Task (as described in Literature Reference No. 10). With respect to pilot training, these tasks were modified in order to adhere to the context of a specific flight simulator, and assets from the flight simulator software were adapted to provide the visual and graphics stimuli of the cognitive tests (an example of which is depicted in FIG. 5). More specifically, FIG. 5 provides an example of a cognitive testing procedure (for a cognitive task). The top of FIG. 5 provides an example of a situation awareness test 500 using flight-simulator specific cues, while the bottom depicts an n-Back task 502 utilizing imagery similar to that seen on simulator navigational maps.

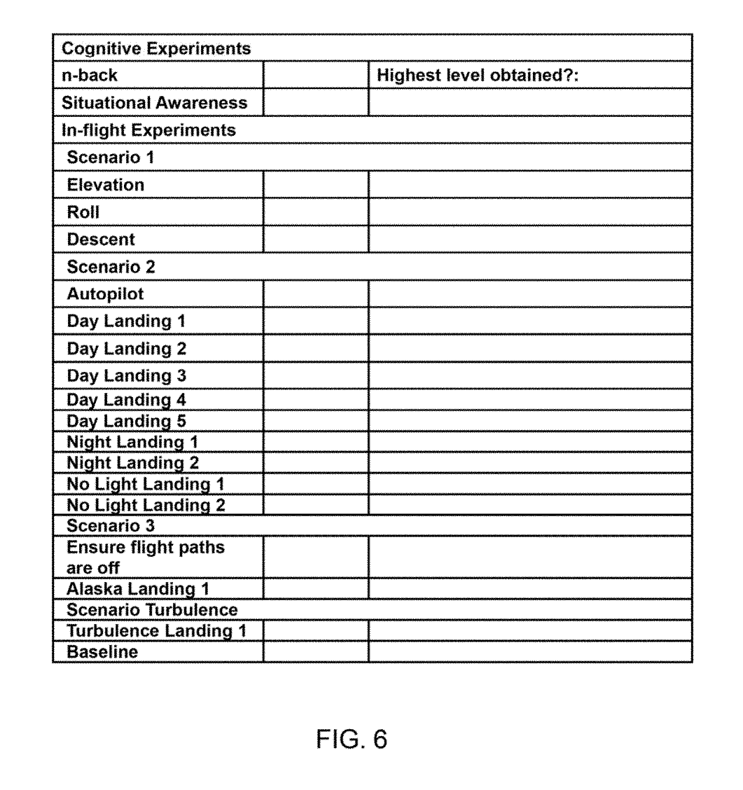

Referring again to FIG. 4, the task component 402 phase of testing tasks subjects with performing basic flight maneuvers that are thought to be prerequisites to performing more complex flight skills and the execution of problem-solving or improvisational actions reflective of familiarity with flight dynamics and control. It should be understood that if the system is implemented in a domain other than pilot training, the selected tasks and maneuvers are altered accordingly. However, with respect to flight, the task component 402 tasks were adapted from input from expert pilots and basic flight instruction as described by flight certification organizations. Non-limiting examples of such tasks include 1) altitude change/maintenance, 2) azimuth change/maintenance, 3) maintenance of vertical speed, and 4) flight during inclement weather (all of which can be monitored and assessed the performance values). An example of the protocol is provided in the table as illustrated in FIG. 6.

Referring again to FIG. 4, the complex real-world skills 404 phase of performance assessment concerns tasks that represent a complex synthesis of fundamental flight skills to practical aviation tasks. Or in other domains, fundamental skills to practical tasks. In order to maintain experimental control over the many variables inherent in an actual, real-world scenario, a test was performed that focused primarily on the landing task, in which subjects were tasked with landing at a particular vertical speed onto a target area of runway in a variety of environments. These environments were of varying difficulty, from “easy” scenarios consisting of wide runway areas and clear visibility to “difficult” scenarios in which the terrain required significant navigation, or visibility was limited to force reliance on aircraft instruments and aviation experience. The particular scenarios presented to subjects are outlined in the table illustrated in FIG. 6. Given these assessment sources, the system collects multimodal data from recording instruments and the flight simulator itself, which provides diagnostic and predictive data from which one could assess expertise/skill levels of pilots as they proceeded through their training program. These will be outlined in the next section.

As noted above, the behavioral and neurological data is assessed 306 to determine the performance parameters of the subject and assess if the subject has achieved the selected training goal. In order to do so, data is collected in two main areas, including neurological data 406 and behavioral data 408.

The neurological data 406 is brain-state data that is collected in order to determine the neurophysiological state of each subject as they proceed through the training and testing regime. The neurological data 406 can be collected using one or more suitable modalities for determining a neurophysiological state. Two non-limiting examples of such modalities are as follows:

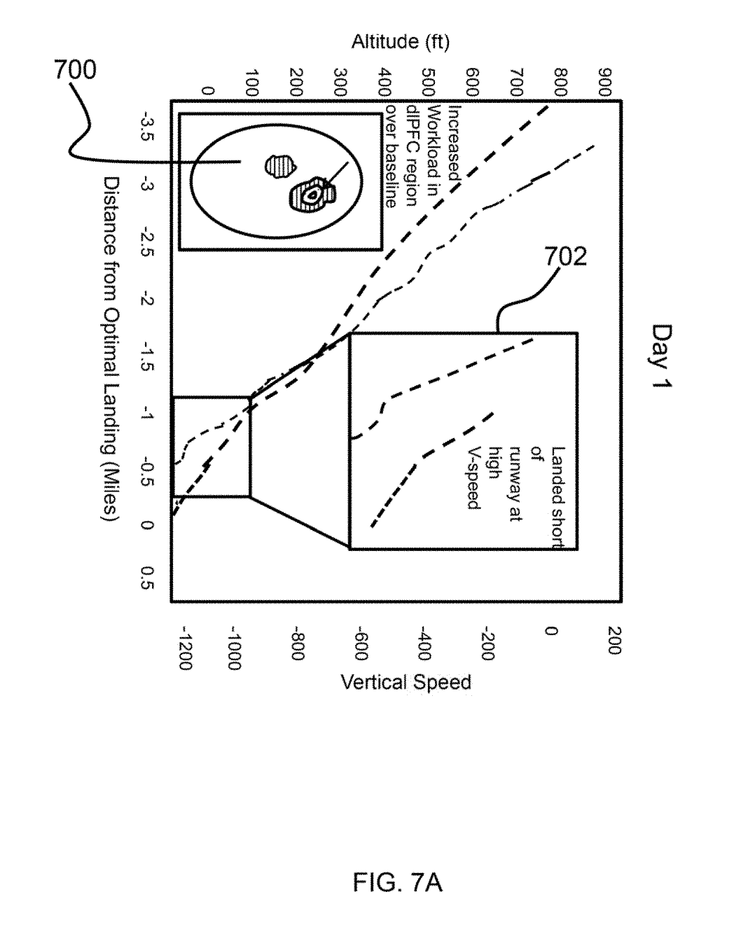

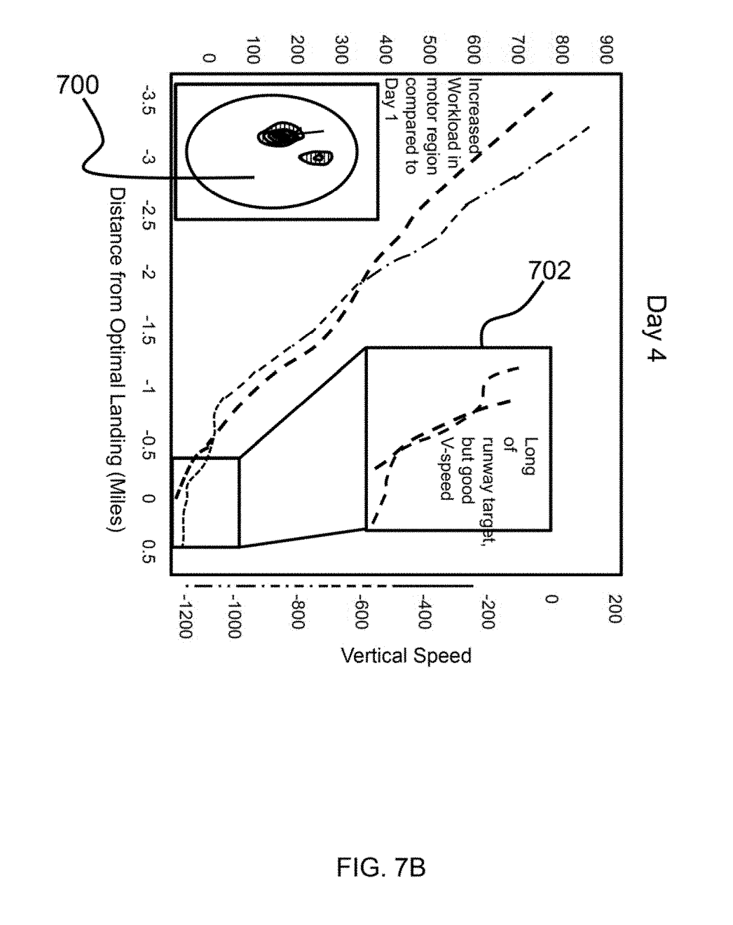

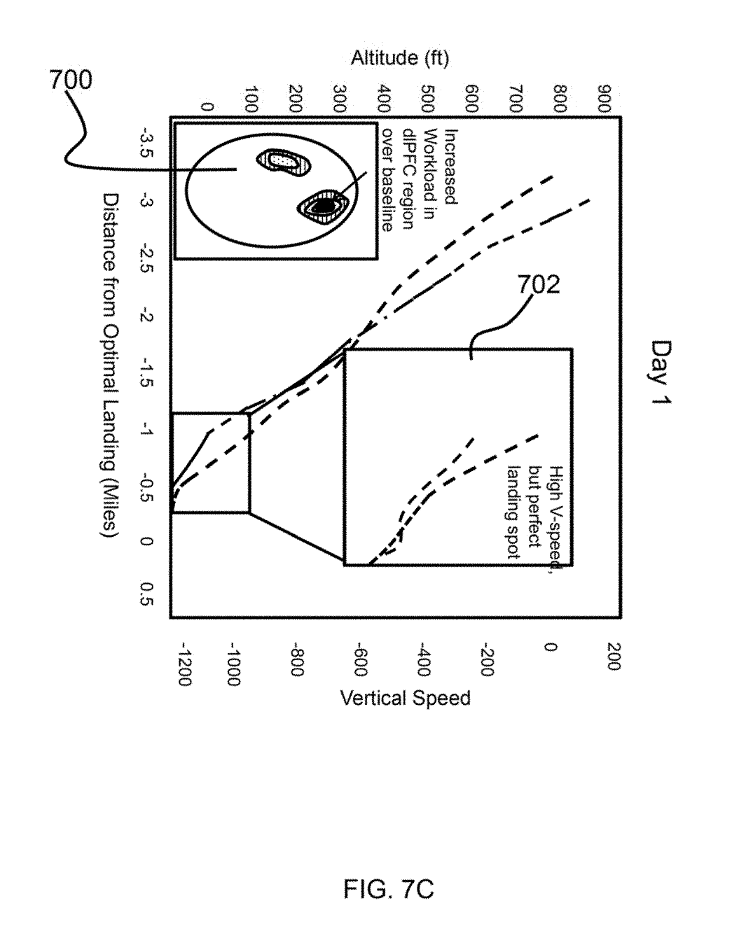

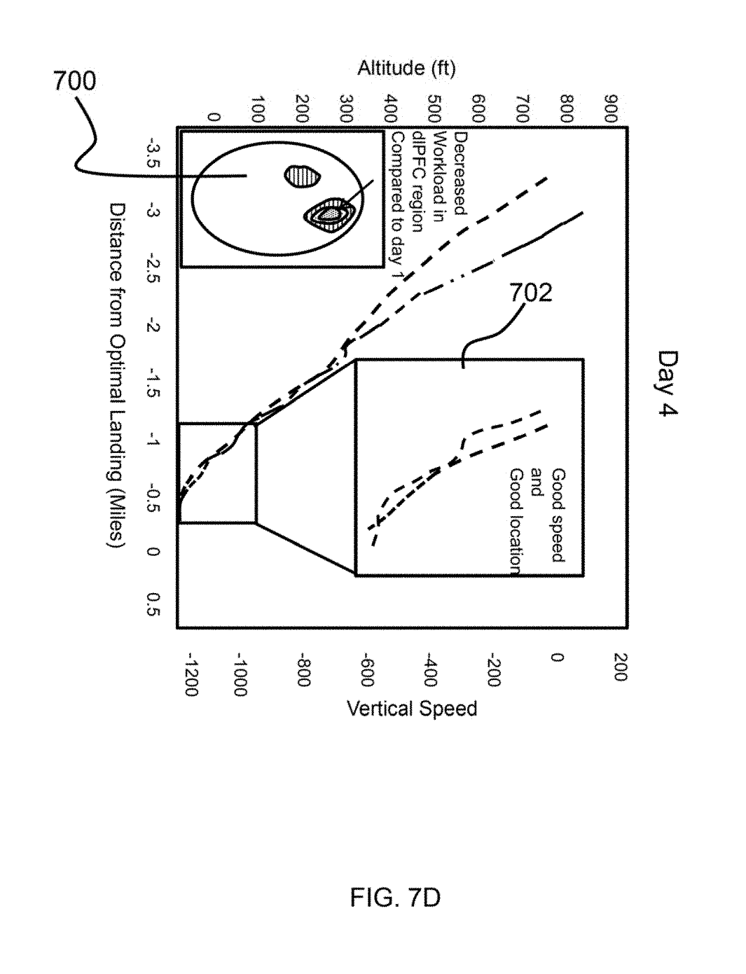

1. Functional Near-infrared Spectroscopy (fNIRS). fNIRS is a brain imaging technology that measures brain activity through monitoring of blood oxygen level dependent (BOLD) contrast, similar to functional magnetic resonance imaging (fMRI). The advantage of fNIRS technology is the portability and cost of the devices, which allows an administrator to observe high-spatial-resolution brain activity in subjects operating a flight simulator. This is impossible with large-scale fMRI devices. This paradigm allows the system (or an operator) to identify active regions of the brain in real-time throughout the subject's assessment period, which provided hard, quantitative data on brain states during various cognitive and behavioral events. One such example is shown in FIGs, 7A through 713, in which the landing performance of stimulated (shown in FIGS. 7C and 7D) and =stimulated (shown in FIGS. 7A and 7B) subjects were compared across the training period. Note that stimulated subjects were subjected to the electrode stimulations referenced above. The fNIRS imagery 700 show that distinct brain-state changes are associated with the increased landing performance of the stimulated group. 2. Electroencephalogram (EEG). EEG is a brain imaging modality that measures aggregate neural activity through electrical potentials recorded from the scalp. Because the signals are recorded through a significant portion of tissue, this results in low-spatial resolution imagery of brain activity; however, because EEG records electrical potentials of neural populations directly, these signals are of high spatial resolution and EEG can provide accurate information about the temporal dynamics of neuronal populations as well as indicate shifts in large-scale frequency activity of the brain. The latter data is well characterized in the literature as indicators of changing brain, states. This data can be leveraged to identify particular activity patterns of the entire brain that are associated with task proficiency and behavioral load; a subject flying in calm vs. turbulent weather shows significant increases in high gamma (60-100 Hz) brain activity during periods of high turbulence. By observing EEG power spectra throughout the course of behavior, the system (or an operator) can begin to pair brain-state indicators with aspects of skill use and retrieval, which provides a well-defined physiological indicator from which to make predictions and diagnostics.

The behavioral data 408 is behavior that can be collected using one or more suitable modalities for determining a subject's behavior. Two non-limiting examples of such modalities are as follows:

1. Performance Data. Measurable performance data, such as flight data from a simulator, is recorded constantly throughout trial. Flight data, for example, provides information about the control inputs used, the status of the aircraft, the active'environmental variables, and geographical/physical information about the aircraft in relation to the simulated world. This data is used to generate detailed information about the specific maneuvers undertaken by subjects during flight. An example of this can be seen in FIGS. 7A and 7B, in which the specific landing location and flight trajectory of stimulated (shown in FIG. 7B) and unstimulated (shown in FIG. 7A) subjects can be compared against “ideal” scenario targets. Particular subcomponents of landing (such as “vertical speed”) can be isolated as problem areas identified as potential foci of supplemental training, and training can be optimized in order to refine particular skills in a continuous fashion. As noted above and as illustrated in FIGS. 7A and 7B, fNIRS is correlated with high-specificity flight behavioral data, associating spatial patterns of brain activation with behavioral ends.

2. Survey statistics. Questionnaires are given to each subject in order to determine qualitative aspects of training that have been identified in skill acquisition literature as critical. These include factors such as motivation and enjoyment, which have been identified by investigators as integral components that impact the learning of complex tasks (see, for example, Literature Reference No. 5).

These data, examined as a whole, are a significant advance from the state-of the-art in terms of assessment of expert piloting skill and performance, and allow training programs and augmentation strategies to be applied in a more precise, specific, and optimized fashion. Further and as noted above, although pilot training is provided as an example, it should be understood that the present invention is not intended to be limited thereto and can be applied to any training program in which a subject performs tasks during the training process. For example, other training might involve ground vehicle operation (cars, forklifts, trucks) or machine operation (industrial machines, typing).

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

What is claimed is:

1. A system for training and assessment, the system comprising:
one or more processors and a memory, the memory being a non-transitory computer-readable medium having executable instructions encoded thereon, such that upon execution of the instructions, the one or more processors perform operations of:
classifying a subject's baseline brain state and behavioral performance;
subjecting the subject to neurological stimulation while the subject performs specified tasks; and
assessing behavioral data to determine if the subject has achieved training goals.

2. The system as set, forth in claim 1, wherein in assessing behavioral data to determine if the subject has achieved the training goals, if the individual has not achieved the training goals, then performing operations of:
reviewing neurological data to identify activation states and values of the neurological stimulation that resulted in increased performance values from the baseline behavioral performance;
adjusting the activation states and values of the neurological stimulation to match those that resulted in increased performance values; and
repeating the operations of subjecting the subject to neurological stimulation, assessing behavioral data, and reviewing the neurological data to identify activation states and values until the subject has achieved the training goals.

3. The system as set forth in claim 2, wherein in reviewing neurological data to identify activation states and values of the neurological stimulation, the neurological data is at least one of functional near-infrared spectroscopy imagery and electroencephalogram data.

4. The system as set forth in claim 3, further comprising an operation of assessing training goals to specify which tasks the subject is to perform and a desired level of performance, and wherein in assessing behavioral data, the behavioral data includes performance data.

5. The system as set forth in claim 4, wherein the system for training and assessment is a pilot training and assessment system, such that when the subject performs the specified tasks, the specified tasks are performed in a flight simulator, with the behavioral data being flight data as recorded by the flight simulator.

6. The system as set forth in claim 5, further comprising a headgear having a plurality of sensors and stimulators.

7. The system as set forth in claim 1, wherein in assessing behavioral data to determine if the subject has achieved training goals, if the individual has not achieved the training goals, then performing operations of:
reviewing neurological data to identify activation states and values of the neurological stimulation that resulted in increased performance values from the baseline behavioral performance;
adjusting the activation states and values of the neurological stimulation to match those that resulted in increased performance values; and
repeating the operations of subjecting the subject to neurological stimulation, assessing behavioral data, and reviewing the neurological data to identity activation states and values until the subject has achieved the training goals.

8. The system as set forth in claim 7, wherein in reviewing neurological data to identify activation states and values of the neurological stimulation, the neurological data is at least one of functional near-infrared spectroscopy imagery and electroencephalogram data.

9. The system as set forth in claim 1, further comprising an operation of assessing training goals to specify which tasks the subject is to perform and a desired level of performance, and wherein in assessing behavioral data, the behavioral data includes performance data.

10. The system as set forth in claim 1, wherein the system for training and assessment is a pilot training and assessment system, such that when the subject performs the specified tasks, the specified tasks are performed in a flight simulator, with the behavioral data being flight data as recorded by the flight simulator.

11. The system as set forth in claim 1, further comprising a headgear having a plurality of sensors and stimulators.

12. A computer program product for training and assessment, the computer program product comprising:
a non-transitory computer-readable medium having executable instructions encoded thereon, such that upon execution of the instructions by one or more processors, the one or more processors perform operations of:
classifying a subject's baseline brain state and behavioral performance;
subjecting the subject to neurological stimulation while the subject performs specified tasks; and
assessing behavioral data to determine if the subject has achieved the training goals.

13. The computer program product as set forth in claim 12, wherein in assessing behavioral data to determine if the subject has achieved the training goals, if the individual has not achieved the training goals, then performing operations of:
reviewing neurological data to identify activation states and values of the neurological stimulation that resulted in increased performance values from the baseline behavioral performance;
adjusting the activation states and values of the neurological stimulation to match those that resulted in increased performance values; and
repeating the operations of subjecting the subject to neurological stimulation, assessing behavioral data, and reviewing the neurological data to identify activation states and values until the subject has achieved the training goals.

14. The computer program product as set forth in claim 13, wherein in reviewing neurological data to identify activation states and values of the neurological stimulation, the neurological data is at least one of functional near-infrared spectroscopy imagery and electroencephalogram data.

15. The computer program product as set forth in claim 13, wherein subjecting the subject to neurological stimulation while the subject performs specified tasks, stimulation is provided to the subject by sending direct currents through stimulators positioned against the subject's scalp, and wherein in reviewing neurological data to identify activation states, neurological data is obtained by sensors portioned against the subject's scalp.

16. The computer program product as set forth in claim 12, wherein in assessing behavioral data to determine if the subject has achieved the training goals, if the subject has achieved the selected goal, then stopping.

17. The computer program product as set forth in claim 12, further comprising instructions for causing the one or more processors to perform operation of assessing training goals to specify which tasks the subject is to perform and a desired level of performance, and wherein in assessing behavioral data, the behavioral data includes performance data.

18. The computer program product as set forth in claim 12, wherein in subjecting the subject to neurological stimulation while the subject performs specified tasks, the training and assessment is directed to pilot training and assessment, such that when the subject performs the specified tasks, the specified tasks are performed in a flight simulator, with the behavioral data being flight data as recorded by the flight simulator.

19. A computer implemented method for training and assessment, the computer implemented method comprising an act of:
causing one or more processers to execute instructions encoded on a non-transitory computer-readable medium, such that upon execution, the one or more processors perform operations of:
classifying a subject's baseline brain state and behavioral performance;
subjecting the subject to neurological stimulation while the subject performs specified tasks; and
assessing behavioral data to determine if the subject has achieved the training goals.

20. The computer implemented method as set forth in claim 19, wherein in assessing behavioral data to determine if the subject has achieved the training goals, if the individual has not achieved the training goals, then performing operations of:
reviewing neurological data to identify activation states and values of the neurological stimulation that resulted in increased performance values from the baseline behavioral performance;
adjusting the activation states and values of the neurological stimulation to match those that resulted in increased performance values; and
repeating the operations of subjecting the subject to neurological stimulation, assessing behavioral data, and reviewing the neurological data to identify activation states and values until the subject has achieved the training goals.

21. The computer implemented method as set forth in claim 20, wherein in reviewing neurological data to identify activation states and values of the neurological stimulation, the neurological data is at least one of functional near-infrared spectroscopy imagery and electroencephalogram data.

22. The computer implemented method as set forth in claim 20, wherein subjecting the subject to neurological stimulation while the subject performs specified tasks, stimulation is provided to the subject by sending direct currents through stimulators positioned against the subject's scalp, and wherein in reviewing neurological data to identify activation states, neurological data is obtained by sensors portioned against the subject's scalp.

23. The computer implemented method as set forth in claim 20, further comprising an operation of assessing training goals to specify which tasks the subject is to perform and a desired level of performance, and wherein in assessing behavioral data, the behavioral data includes performance data.

24. The computer implemented method as set forth in claim 19, wherein in subjecting the subject to neurological stimulation while the subject performs specified tasks, the training and assessment is directed to pilot training and assessment, such that when the subject performs the specified tasks, the specified tasks are performed in a flight simulator, with the behavioral data being flight data as recorded by the flight simulator.
